(19) United States
(12) Patent Application Publication
(10) Pub. No.: US 2016/0297700 A1
(43) Pub. Date: Oct. 13, 2016

(54) PSYCHROPHILIC ANAEROBIC DIGESTION OF HIGH SOLIDS CONTENT

(71) Applicant: HER MAJESTY THE QUEEN IN RIGHT OF CANADA, AS PRESENTED BY THE MINISTER OF AGRICULTURE AND, Sherbrooke (CA)

(72) Inventor: Daniel I. MASSÉ, Sherbrooke (CA)

(73) Assignee: HER MAJESTY THE QUEEN IN RIGHT OF CANADA, AS PRESENTED BY THE MINISTER OF AGRICULTURE AND, Sherbrooke, QC (CA)

(21) Appl. No.: 15/098,747

(22) PCT Filed: Oct. 14, 2014
(86) PCT No.: PCT/CA2014/050988

§ 371 Date: Apr. 14, 2016

Related U.S. Application Data
(60) Provisional application No. 61/892,518, filed on Oct. 18, 2013.

(51) Int. Cl. C02F 11/04; C12P 5/02; C05F 9/00; C05F 11/00; C02F 3/28; C05F 3/00

(52) U.S. Cl. CPC C02F 11/04; C02F 3/28; C05F 3/00; C05F 9/00; C05F 11/00; C12P 5/023; C02F 2103/20

(57) ABSTRACT

The present description relates to a process for the psychrophilic anaerobic digestion of high organic solids content waste, such as farm manure or municipal waste, comprising the steps of contacting the high organic solids content waste to an inoculum comprising anaerobic bacteria in a digester and reacting the high organic solids content waste with the inoculum at a temperature below 25° C. to allow digestion of the high organic solids content waste.

### TECHNICAL FIELD

The present description relates to a psychrophilic anaerobic digestion process of high organic solids content waste.

### BACKGROUND ART

Livestock manures produced by Canadian and USA livestock represent between 75 and 86% of the total manure production (Wen et al., 2004, Bioresource Technology, 91: 31-39). Fresh cow feces has a total solids content (TS) of about 12-14% and when straw is used as bedding material then the total solids ranges between 15% and 25%, depending on the amount of bedding used. Processing organic waste from farms and livestock industries using anaerobic digestion is under intensive research to extract renewable energy and reduce the environmental footprint of the livestock industry. Stabilizing such waste in wet anaerobic digestion processes requires dilution to decrease the solids content for liquid handling and processing. Dilution results in substantially larger digester volume. An alternative process with high solids content organic wastes is the dry anaerobic digestion.

Around 40-50% of the volatile solids (VS) in dairy manure is biodegradable lingocellulosics biomass containing reduced carbon which can be converted to CH₄ (Abbassi-Guendouz et al., 2012, Bioresour Technol, 111: 55-61). Mesophilic or thermophilic dry anaerobic digesters (DAD) of agricultural wastes with high solids content is relatively a new biotechnology (Ahn et al., 2010, Applied Biochemistry and Biotechnology, 160: 969-975; Kusch et al., 2008, Bioresource Technology, 99: 1280-1292). Suitability and economic feasibility of on-farm mesophilic DAD for solid manure, crop residues, spoiled hay and silage, and energy crops has been studies extensively by Schafer et al. (2006, “Dry anaerobic digestion of organic residues on farm—a feasibility study”, Agrifood Research Reports 77, MIT Agrifood Research Finland).

Converting complex and high-solids lignocellulosic substrates such as cow manure and bedding to methane is challenged by mass transfer and biological kinetic limitations because microorganisms mediating the anaerobic digestion reactions depend on the flow of intermediate by-products from one trophic group to another (McInerney & Beaty, 1988, Canadian Journal of Microbiology, 34 487-493).

Lack of research and commercial development of on-farm DAD is behind its current low acceptance. There is thus still a need to be provided with a robust and low cost DAD process for on-farms applications to digest livestock manures.

### SUMMARY

In accordance with the present description there is now provided a process for the psychrophilic anaerobic digestion of high organic solids content waste comprising the steps of contacting the high organic solids content waste to an inoculum comprising anaerobic bacteria in a digester and reacting the high organic solids content waste with the inoculum at a temperature below 25° C. to allow digestion of the high organic solids content waste.

In an embodiment, the high organic solids content waste is reacted with the inoculum at a temperature of between 10 to 25° C.

In another embodiment, the high organic solids content waste is reacted with the inoculum at a temperature of 20° C.

In a further embodiment, the high organic solids content waste comprises between 12-45% of total solids content.

In another embodiment, the high organic solids content waste is animal manure, energy crops, agri-food or municipal wastes.

In a particular embodiment, the animal manure is farm waste.

In another embodiment, the farm waste comprises a high fibrous content.

In another embodiment, the farm waste is dairy manure, beef manure, poultry manure, spoiled hay, silage or solid fraction of swine manure.

In a further embodiment, the farm waste is cow manure.

In another embodiment, the animal manure comprises cellulose, hemicellulose, lignin, fat and protein or a mixture thereof.

In another embodiment, the process described herein further comprises the step of feeding the digester with inoculum from the same digester or a separate silo.

In an embodiment, the inoculum is feed continuously from the separate silo into the digester.

In a further embodiment, the inoculum is recuperated at the end of the digestion

In another embodiment, the digester is a batch reactor, a sequential batch reactor or a plug flow digester.

In another embodiment, methane is recuperated during digestion of the high organic solids content waste.

In a further embodiment, a fertilizer is recuperated from the digester after digestion of the high organic solids content waste.

In another embodiment, the high organic solids content waste is digested within 21 days or less.

### BRIEF DESCRIPTION OF THE DRAWINGS

Reference will now be made to the accompanying drawings, showing by way of illustration, a preferred embodiment thereof, and in which:

FIG. 1 illustrates specific methane yield profiles for cow feces and wheat straw psychrophilic dry anaerobic digestion as described herein.

FIG. 2 illustrates volatile fatty acids profiles for cow feces and wheat straw psychrophilic dry anaerobic digestion, wherein A-Acetic, B-propionic, and C-Butyric acids are measured.

FIG. 3 illustrates the pH profile for cow feces and wheat straw psychrophilic dry anaerobic digestion as described herein.

FIG. 4 illustrates the total solid profile for the cow feces and wheat straw psychrophilic dry anaerobic digestion as described herein.

FIG. 5 illustrates the specific methane yield profiles for the cow feces and wheat straw (27% TS) psychrophilic anaerobic digestion.

FIG. 6 illustrates the specific methane yield profiles for the high-rate psychrophilic dry anaerobic digestion of cow feces and wheat straw (27% TS).

FIG. 7 illustrates the specific methane yield profiles for psychrophilic dry anaerobic digestion of cow feces and wheat straw (35% TS) at OLR of 3 g TCOD kg⁻¹ d⁻¹.

FIG. 8 illustrates the specific methane yield profiles for psychrophilic dry anaerobic digestion of cow feces and wheat straw (35% TS) at OLR of 4.0 and 5.0 g TCOD kg⁻¹ d⁻¹.

FIG. 9 illustrates the specific methane yield profiles for psychrophilic dry anaerobic digestion of (A) corn silage; (B) barley silage; and (C) Grass silage with and without cow feces at OLR of 3.0 g TCOD kg⁻¹ d⁻¹.

FIG. 10 illustrates the results of long term (210 days experiment comprising 10 successive cycles) of psychrophilic (20° C.) dry anaerobic digestion (PDAD) of cow feces and wheat straw at feed TS of 27% in laboratory scale sequence batch reactor operated at increasing organic loading rate 7 and 8.0 g TCOD kg⁻¹ d⁻¹.

### DETAILED DESCRIPTION

It is provided a psychrophilic anaerobic digestion process of high organic solids content waste, such as animal manure, that can be integrated for example in a farm waste management to potentially increase farmers income while reducing the environmental footprint of the operation.

It is encompassed herein that the process described herein can be used to not only digest animal manure such as farm waste, but also energy crops, agri-food or municipal wastes.

Increasing the total solids of the substrate fed to dry anaerobic digesters is an engineering design objective to decrease the construction cost by reducing the bioreactor volume as well as the volume of the bioreactor's effluent to store and land apply.

The psychrophilic anaerobic digestion (PAD) in sequential batch reactor (SBR), developed at Agriculture and Agri-Food, Dairy and swine Research and Development Centre (DSRDC) in Sherbrooke, Quebec-Canada for the stabilization of agricultural wastes, successfully reduces odors, decreases the organic pollution load by more than 70% (Masse et al., 1996, Canadian Jounrla of Civil Engineering, 23: 1285-1294), produces high quality biogas, significantly diminishes pathogens survival (Masse et al., 2011, Borescource Technology, 102: 641-646), and improves the agronomic value of digestate (Masse et al., 2007, Bioresource Technology, 98: 2819-2823).

The process offers the competitive advantages of great stability, robustness, maximum performance, and minimum supervision. Moreover, less energy is required to maintain the temperature in the digester as compared to mesophilic and thermophilic anaerobic digestion. The process uses bacteria adapted to thrive at low temperature (Dhaked et al., 2010, Waste Management, 30: 2490-2496) and digest organic substrates with TS contents lower than 12%, such as swine manure. Low temperature wet anaerobic digestion provides a unique, very stable and cost effective process for digesting liquid swine manure.

Canadian patent no. 2,138,091 describes a psychrophilic anaerobic digestion of animal manure slurry in intermittently fed sequencing batch reactors. The manure slurry being digested is liquid swine and dairy manures (low solid content <10%) under solid or semi-solid state (1-10% TS) which has a very low content in fibrous material compared to high solids content manure, such as cow, poultry or generally dairy manure for example. A similar psychrophilic anaerobic digestion process as described in Canadian patent no. 2,138,091 has also been demonstrated to be able to remove hydrogen sulphide content from the biogas produced during digestion (see WO 2012/061933) and to degrade prions contained in the starting material to be digested (see WO 2011/152885).

Acclimatized anaerobic sludge contained in reactors used in the liquid PAD process described in Canadian patent no. 2,138,091, WO 2012/061933 or WO 2011/152885, were unsuccessful in efficiently digesting manure with high solids content. The microbial consortium was not successful in processing high solids dairy manure even with a long treatment cycle of 350 days.

A PAD process is described herein for the first time for agricultural wastes with high solids content such as dairy manure with bedding. Before the present disclosure, with the addition of bedding, the manure is usually under solid or semi-solid state (15-20% TS). Such substrate must be primarily diluted before treatment in order to decrease its TS content for liquid handling (El-Mashad et al., 2004, Bioresource Technology, 95: 191-201). The dilution reduces the solids content (TS value), nutrient concentration, increases the volume of substrate to be treated, thus increases the bioreactor volume and the quantity of bioreactor effluent to store and dispose.

This is the first report on successful long-term psychrophilic (20° C.) dry anaerobic digestion of high solids content, such as cow feces with wheat straw at 27% total solids. It is demonstrated the feasibility of dry anaerobic digestion (DAD) of cow feces and wheat straw in long-term operation in a sequencing batch reactor.

It is thus disclosed a process for the psychrophilic anaerobic digestion of high organic solids content waste comprising the steps of contacting the high organic solids content waste to an inoculum comprising anaerobic bacteria in a digester and reacting the high organic solids content waste with the inoculum at a temperature below 25° C., representing psychrophilic conditions.

Psychrophilic conditions are known to reflect bacteria activity at a temperature of about 10° C. to about 25° C.

The high organic solids content waste encompassed herein that can be digested are not only farm manure with high fibrous content, such as dairy manure (cow manure), beef manure, poultry manure and agri-food waste, etc. for example, but also municipal waste with high solids content. High organic solids content waste are generally intended as waste having between 12-45% TS.

It is demonstrated herein that a treatment cycle length of 21-day provided improved result than published prior studies with substantially longer treatment cycle length (see Table 2). Also encompassed herein a longer treatment cycle length (>21 day) to provide more biogas.

It is demonstrated the performance of psychrophilic dry anaerobic digestion of dairy cow manure and wheat straw at 27% in long term operation. The 27% TS in feed was exemplified because cattle in barn with straw litter produce manure with a TS content up to 27% TS (Demirer and Chen, 2008, Waste Manag, 28: 112-119). This is the first report on successful psychrophilic (20° C.) dry anaerobic digestion of cow feces plus wheat straw at 27% total solids in long-term operation (273 days). An average specific methane yield (SMY) of 182.9±16.9 _{N}L CH₄ kg⁻¹ VS fed during 12 successive cycles (273 days) has been achieved for feed total solids of 27% at organic loading rate 3.0 g TCOD kg⁻¹ culture day⁻¹ and hydraulic retention time of 21 days. A maximum SMY of 219.2±18 _{N}L CH₄ kg⁻¹ VS fed (177.1±44 _{N}L CH₄ kg⁻¹ COD fed) with a maximum CH₄ production rate of 10.2±0.8 _{N}L CH₄ kg⁻¹ VS day⁻¹ have been accomplished. The low accumulation of volatile fatty acids during each treatment cycle indicated that hydrolysis was the reaction limiting step. The SMY and CH₄ production rate increased gradually and stabilized during the long-term operation. The results indicate that dry anaerobic digestion of dairy cow manure and wheat straw at 27% and hydraulic retention time (HRT) of 21 is feasible in sequential batch reactor for example, but not limited to, with a solids retention time of 6 times the HRT. The recuperated methane contained in biogas represent a potent greenhouse gas, which can be a clean a renewable source of energy. Recovered biogas can thus be used to generate electricity in internal combustion engines (Lusk, 1998, “Methane recovery from animal manures—The current opportunities casebook”, Resource Development Associates Wash., D.C., National Renewable Energy Laboratory; Savery and Cruzan, 1972, J Water Pollution Control Federation, 44: 2349-2354).

The process described herein also allows recuperating inoculum at the end of the digestion process in order to be stocked in a silo or reuse in the digester in a semi-continuous or continuous process. As exemplified herein, the inoculum from the same digester is used. At the end of the treatment cycle a fraction of the bioreactor solid effluent (inoculum) was premixed with the organic substrate prior reloading the reactor. The solid inoculum could be diluted and stored in a separate silo and reused to inoculate a new batch of solid substrate in the bioreactor. It is recirculated from the separate silo into the digester.

Fertilizer can also be recuperated at the end of the process. The fertilizer can then be used to supplement farm fields for example.

When solid inoculum is used, only a fraction of the bioreactor content (inoculum) is recuperated and used to inoculate the next batch of solids substrate. When liquid inoculum (diluted inoculum) is stored in a separate silo, as much inoculum as possible is recuperated (ideally 100%).

No mixing and heating mechanism are needed in the reactor, increasing the net energy yield per unit volume of the reactor. Huebel and Dixhorn (2006, Water Environment Foundation, 407-414) reported that 564.4 mol CH₄ m⁻³ substrate day⁻¹ was required to maintain the temperature of an anaerobic digester at 37° C. when handling a substrate of 0.96% TS with an HRT of 20 days. Furthermore, increasing the total solids from 18 to 27% means around 35% reduction in the required volume of the bioreactor. Investment cost decreases by 70% when the reactor volume is reduced by 50%. Therefore, the PAD described herein offers the advantage of the combined saving in cost of construction and energy expenses of heating and mixing. Furthermore, generally a DAD process reduces the quantity of liquid effluent discharged from the reactor by 75% (Luning et al., 2003, Wat Sci Technol, 48: 15-20); resulting in more saving during post-treatment (handling, dewatering, and disposal) facilities and operation from using a PAD process as described herein.

The percent of H₂S in the biogas was less than 0.06% in all samples of gas analyzed during the successive cycles. The cow feces fed during the whole duration of the experiment contained fibers composed of cellulose (23.61%), hemicellulose (18.71%) and lignin (11.3%). Similarly, wheat straw fibers composed of cellulose (38.61%), hemicellulose (25.14%) and lignin (7.3%).

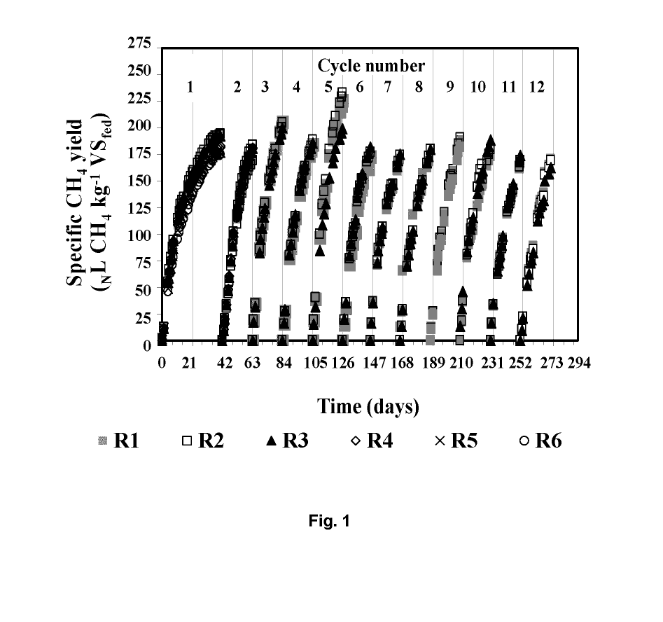

The stability and the performance of psychrophilic anaerobic bioreactors were evaluated in 12 successive cycles (273 days). The profiles of methane production expressed as specific methane yield (SMY) in the replicate bioreactors are shown in FIG. 1. The maximum SMYs calculated during the successive cycles are given in Table 1.

TABLE 1
Rate and specific methane yield for the psychrophilic
anaerobic digestion of cow feces and wheat straw (27% TS)

| | | | | | Rate of CH₄ |
|---|---|---|---|---|---|
| Retention | | TS | SMY | SMY | production |
| time | Cycle | (%) | (_{N}L CH₄ kg⁻¹ VS) | (_{N}L CH₄ kg⁻¹ TCOD) | (_{N}L CH₄ kg⁻¹ VS d⁻¹) |
| 21 | 1 | 27 | 148.6 ± 8 | 114.5 ± 6.4 | 7.1 ± 0.4 |
| 42 | 1 | 27 | 184.1 ± 8 | 142.3 ± 11.1 | 4.3 ± 0.2 |
| 21 | 2 | 27 | 178.6 ± 5 | 131.4 ± 3.5 | 8.5 ± 0.2 |
| 21 | 3 | 27 | 203.4 ± 3 | 156.4 ± 2.6 | 9.6 ± 0.1 |
| 21 | 4 | 27 | 186.1 ± 2 | 146.3 ± 1.4 | 8.8 ± 0.1 |
| 21 | 5 | 27 | 219.2 ± 18 | 155.1 ± 12.7 | 10.4 ± 0.6 |
| 21 | 6 | 27 | 177.2 ± 4.3 | 130.5 ± 3.2 | 8.4 ± 0.8 |
| 21 | 7 | 27 | 172.7 ± 2.6 | 127.2 ± 1.9 | 8.2 ± 0.0 |
| 21 | 8 | 27 | 175.8 ± 6.5 | 130.5 ± 4.8 | 8.4 ± 0.3 |
| 21 | 9 | 27 | 186.7 ± 4.4 | 137.8 ± 3.9 | 8.9 ± 0.2 |
| 21 | 10 | 27 | 180.7 ± 7.4 | 133.3 ± 5.5 | 8.6 ± 0.4 |
| 21 | 11 | 27 | 169.9 ± 3.8 | 119.1 ± 2.6 | 8.1 ± 0.2 |
| 21 | 12 | 27 | 194.1 ± 4.8 | 126.3 ± 3.2 | 9.2 ± 0.2 |

Note:
Cycle 1 to 4 had 6 replicates bioreactors while cycle 5 to 8 had 3 replicates bioreactors

Based on the total VS fed (cow feces plus wheat straw), the average SMY calculated was 148.6±8, 178.6±5, 203.4±3, 186.1±2, 219.2±18, 177.2±4, 172.7±3, 175.8±7, 186.7±4, 180.7±7, 169.9±4, and 194.1±5 _{N}L CH₄ kg⁻¹ VS fed during the 12 successive cycles for TS 27% and HRT of 21 days (FIG. 1 and Table 1). The variation in the average SMY from cycle to cycle is likely due to the variation in the quality of the cow feces fed. The yield achieved during the first cycle at HRT of 42 days was similar to the overall average yield achieved in the 12 successive cycles at HRT of 21 days which indicates relatively a fast start-up phase of 42 days likely because the culture was well-adapted to the psychrophilic conditions and the high solids content.

The effect of microbial adaptation on the performance of the bioreactors is clearly shown when the SMYs (_{N}L CH₄ kg⁻¹ VS) of the successive cycles are compared to each other; in cycle 1 (148.6±8), cycle 2 (178.6±5), cycle 4 (186.1±2), cycle 9 (186.7±4), and cycle 12 (194.1±5).

Although the organic loading rate (3.0 g TCOD kg⁻¹_{inoc} day⁻¹) and the feed total solids (27%) have been maintained the same during cycles 1 to 10, the SMY at HRT of 21 days increased by 12.5 to 48.5% (average of 24%) during the successive cycles 2 to 10 compared to the SMY obtained during cycle 1. The variation in the calculated SMY among the replicate bioreactors also decreased successively. The slightly large value of the CV during cycle 1 is likely due to the adaptation process while those during cycles 5, 8, and 10 are likely due to variation in the quality of the cow feces fed or experimental error. The overall pattern of performance consistency from cycle to cycle indicates a stable reproducible process during the 273 days of operation.

Usually, two segments are recognized in the SMY curve during batch anaerobic digestion: an initial exponential phase followed by a slowdown phase in CH₄ production for the rest of the incubation period. Cycles 2 to 12 showed only the initial straight line part of the CH₄ production because the HRT was limited to 21 days which means that not all the substrate fed was completely digested during that HRT. However, in the sequential batch reactors the solid retention time (SRT) is different from the HRT. The average SRT is 138.7±5.1 days (Table 4) which allowed the degradation of the substrate fed to a greater extent.

When the first cycle was extended to a second HRT, the SMY increased. The ultimate SMY at 42 days during cycle 1 was 184.4±8 _{N}L CH₄ kg⁻¹ VS fed. This increase in the SMY is related to the contribution of the fibers from wheat straw and cow feces to the biogas production. The dry matter of wheat straw is composed of cellulose (38.61%), hemicellulose (25.14%) and lignin (7.3%). Cellulose requires long retention time to be biodegraded by the anaerobic consortia of microorganisms and its hydrolysis has been shown to be the rate limiting step (Noike et al., 1985, Biotechnology and Bioengineering, 27: 1482-1489) particularly when the substrate is solid or in particulate form (Myint and Nirmalakhandan, 2006, Environmental Engineering Science, 23: 970-980). The increase in the SMY might indicate also that the culture was still adapting. A similar increase in the specific CH₄ production rate can be observed (Table 4). The average specific CH₄ production rate (_{N}L CH₄ kg⁻¹ VS d⁻¹) of the replicate bioreactors was 7.1±0.4 (cycle 1), 8.5±0.2 (cycle 2), 9.6±0.1 (cycle 3), 8.8±0.1 (cycle 4), 10.4±0.6 (cycle 5), 8.4±0.8 (cycle 6), 8.2±0.0 (cycle 7), 8.4±0.3 (cycle 8), 8.9±0.2 (cycle 9), 8.6±0.4 (cycle 10), 8.1±0.2 (cycle 11), and 9.2±0.2 (cycle 12). The trend of methane production rate was increasing successively during the first three cycles, and then stabilized around an overall average of 8.9±0.7 _{N}L CH₄ kg⁻¹ VS d⁻¹ during the last 11 successive cycles (Table 4).

No relevant data is available in the accessible literature on the performance of psychrophilic DAD; therefore, the results have been compared to the performance of mesophilic and thermophilic DAD of various substrates (Table 2).

TABLE 2
Comparative performance of DAD process with prior art

| Substrate | Tem- | | | | Retention | SMY | SMY | |
|---|---|---|---|---|---|---|---|---|
| and | perature | TS | | OLR (g | time | (_{N}L CH₄ kg⁻¹ total | (_{N}L CH₄ kg⁻¹ | |
| inoculum | (° C.) | (%) | SIR | TCOD kg⁻¹_{inoc} day⁻¹) | (days) | VS) | TCOD) | Reference |
| Cow feces and straw | 20 | 27 | 0.18 | 3.0 | 21 | 193.4 ± 7.1 | | Present disclosure |
| Dairy manure and barley | 35 | 13 | | 5.2 | 25 | 159 | 380^{d} | Hills (1980) |
| straw | | | | (g VS I⁻¹_{inoc} day⁻¹) | | (548 L kg⁻¹ | (L kg⁻¹ | |
| | | | | | | VS_{destroyed}) | COD_{destroyed)} | |
| Beef cattle manure | NR | NR | NR | Hydrolysis: 6 | 40 | 85^{c} | NR | Schafer et al. (2006) |
| In two stage (hydrolysis- | | | | Methanogensis: 3 kg | | | | |
| methanogesis) reactors | | | | VS m⁻³ d⁻¹ | | | | |
| Beef manure plus straw | 32 | 18 | NR | 3.2 | 28 | 181^{c} | NR | Schafer et al. (2006) |
| Pig manure with turnip | 35 | 16 | NR | NR | 120 | 122^{c} | NR | Schafer et al. (2006) |
| rape straw and wheat | | | | | | | | |
| straw | | | | | | | | |
| 85% beef manure plus | 35 | 28 | NR | 0.9 | 100 | 227^{c} | NR | Schafer et al. (2006) |
| 15% grass silage | | | | | | | | |
| DM, straw, and oat husk | 38 | 17 | NR | 3.4 | 22 | 160^{c} | NR | Schafer et al. (2006) |
| | | | | 4.1 | | 84 | NR | |
| Fresh HM and straw with | 37 | 20 | 0.2 | NR | 28 | 146 | NR | Kusch et al. (2008) |
| pre-fermented solid HM as | | | | NR | 42 | 175 | NR | |
| inoculum | | | | NR | 72 | 208 | NR | |
| CM:WWS | 35 | 16 | 0.2 | 0.35^{a} | 63 | 328 | 270 | Li et al. (2011a) |
| (2:3 mass ratio) | | 15 | | | 63 | 251 | 210 | |
| | | 16 | | | 63 | 319 | 256 | |
| Aerobically pre-treated SM, | 35 | 28 | NR | 0.28^{b} | 130 | 55 | 55 | Di Maria et al. (2012) |
| agricultural residues | | | | | 120 | 40 | NR | |
| inoculated with CM | | | | | 65 | 22 | NR | |
| SM and SG | 55 | | 0.2 | | | 337^{c} | NR | Ahn et al. (2010) |
| DM and SG | | 15 | 0.2 | NR | 62 | 28^{c} | NR | |
| PM and SG | | | 0.8 | | | 2^{c} | NR | |
| Rice straw and corn stover | 26-28 | 15 | 0.2 | NR | 156 | 346 | NR | Sun et al. (1987) |
| inoculated with (1:1) | | 20 | | NR | 156 | 339 | NR | |
| sewage sludge:pig | | 25 | | NR | 168 | 382 | NR | |
| manure, plus | | 30 | | NR | 198 | 423 | NR | |
| | | 35 | | NR | 198 | 34 | NR | |
| Dairy cattle feces | 35 | 11 | 0.7 | 1.4b | 40 | 161c | NR | Møller et al. (2004) |
| Straw | | | | 1.4b | 40 | 195c | NR | |

The average yield of 182.9±16.9 _{N}L CH₄ kg⁻¹ VS of cow feces and wheat straw (27% TS at OLR 3.0 g TCOD kg⁻¹ culture or 2.12 kg VS_{fed} m⁻³ d⁻¹) obtained in this study after 21 days of psychrophilic (20° C.) incubation during the last 11 successive cycles is greater than the yield 160 _{N}L CH₄ kg⁻¹ VS of dairy manure, straw, and oat husk (TS 17% at OLR of 3.4 kg VS m⁻³ d⁻¹) reported by Schafer et al. (2006, “Dry anaerobic digestion of organic residues on-farm—a feasibility study” Agrifood Research Reports 77, MTT Agrifood Research Finland) for Jarna biogas plant in Sweden which operates at 38° C. and retention time of 22 days. The data reported from Jarna plant is for steady-state condition where the inoculum was adapted to the substrate and the operation condition for long time (3 years); a longer adaptation of psychrophilic culture is expected to increase the yield further.

The SMYs from cow feces and wheat straw at an HRT of 21 days in any of the PAD seven successive cycles (TS 27%) obtained in the experimental study described herein are higher than 28 L CH₄ kg⁻¹ VS of dairy manure and switch grass (15% TS) obtained by Ahn et al. (2010, Applied Biochemistry and Biotechnology, 160: 965-975) during 62 days of thermophilic (55° C.) incubation. The average SMY (182.9±16.9 _{N}L CH₄ kg⁻¹ VS fed) is similar to 181 L CH₄ kg⁻¹ VS of beef manure and straw (TS 18% and OLR of 3.2 kg VS m⁻³ d⁻¹) at 32° C. and retention time of 28 days reported by Schafer et al. (supra) (Table 4). Compared to Schafer et al. results, the 30% increase in the feed total solids in the current study (from 18 to 27%) translates into 35% reduction in the required volume of the bioreactor. Similarly, the 25% reduction in the treatment cycle length (from 28 to 21 days) achieved in this study means an additional 25% reduction in the required volume of the bioreactor. Furthermore, operating at psychrophilic condition reduces the reactor heating expenses.

The high yields (>250 _{N}L CH₄ kg⁻¹ VS fed) reported by Li et al. (2011, International Journal of Physical Sciences, 6: 3679-3688) and Sun et al. (1987, Biological Wastes, 20: 291-302) in Table 4 have been obtained for long retention times (>156 days) and low OLR (0.35 g TCOD kg⁻¹_{inoc} day⁻¹), respectively. Achieving a stable dry anaerobic digestion of cow manure and wheat straw at psychrophilic condition and feed TS of 27% over long-term operation is a significant improvement given that 30% TS has been recently identified as a threshold above which methanogenesis was strongly inhibited at 35° C. (Abbassi-Guendouz et al., 2012, Bioresour Technol, 111: 55-61).

The specific methane yields obtained in this study provide evidence that PAD of cow manure and straw, also defined herein as psychrophilic dry anaerobic digestion (PDAD) is practically feasible at TS 27% and is as efficient as mesophilic DAD given that a well-acclimatized inoculum is developed and maintained.

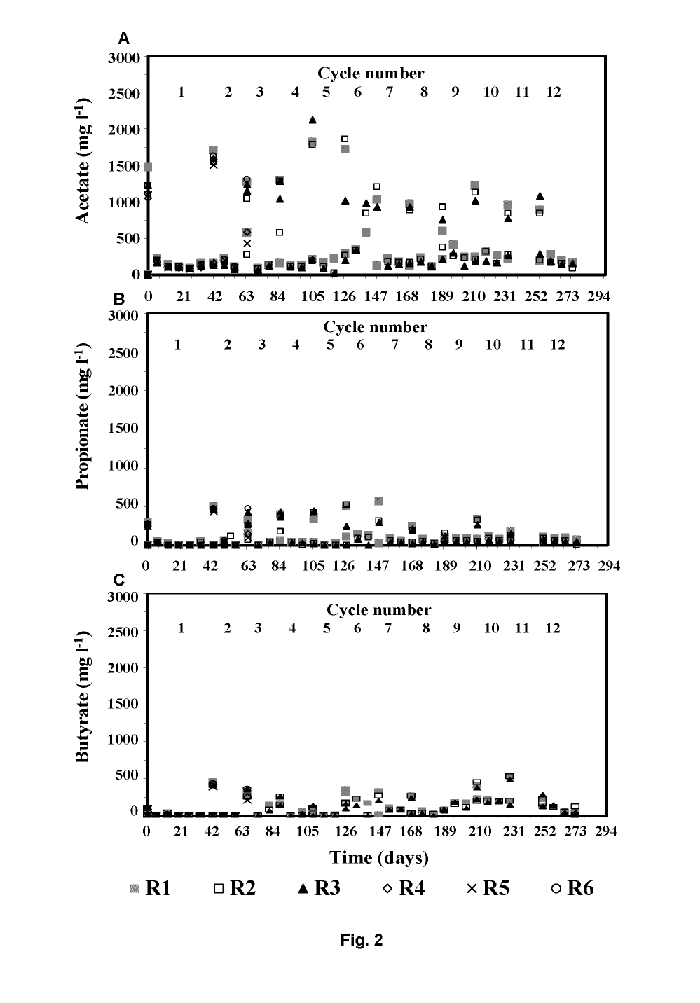

Profiles of acetic, propionic, and butyric acids produced during the successive cycles of PDAD with increasing total solids percent in the feed are shown in FIG. 2. The profiles of VFAs produced in the six replicate bioreactors were almost identical. Throughout the successive cycles, acetic acid concentration peaked immediately after feeding to levels between 1000-2000 mg I⁻¹ but was consumed within a week in all replicate bioreactors and its concentrations were maintained within 100±50 mg I⁻¹ indicating that methanogenesis reaction from acetate was not a rate limiting step. Similarly, propionic acid peaked to levels between 500 and 600 mg I⁻¹ after feedings and was consumed within a week to levels close to the detection limits of the instrument (25±10 mg I⁻¹). The profile of propionic acid in the replicate bioreactors was similar to that of acetic acid. Butyric acid peaked also to levels between 500 and 600 mg I⁻¹ after feedings and was consumed within a week to levels close to the detection limits of the instrument (25±10 mg I⁻¹). The concentrations of other volatile fatty acids (isobutyric-, iso-valeric-, and valeric-acid) were less than 100 mg I⁻¹ immediately after feeding and less than 50 mg I⁻¹ during the remaining time of the successive cycles.

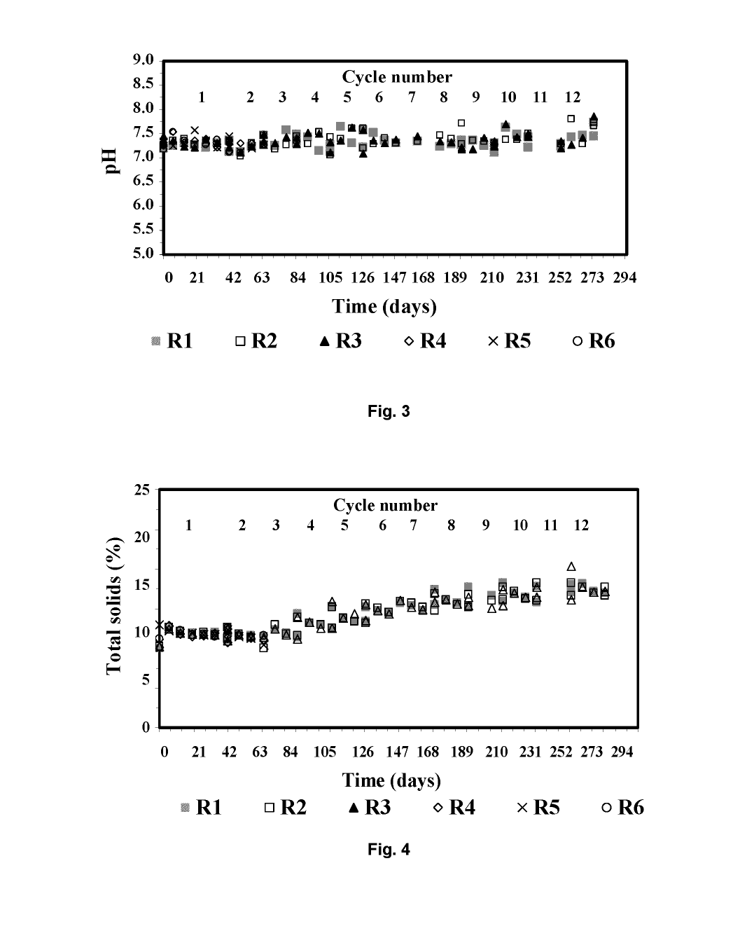

The similar VFAs concentration profiles and SMY in the successive cycles demonstrate that a pseudo steady-state operation has been reached and that acetogenic and methanogenic reactions were well balanced. The relative stability of the pH profile around 7.2±0.2 (FIG. 3) was due to the high alkalinity (8200 mg CaCO₃ I⁻¹) of the mixed liquor and the low concentration of VFAs.

The profiles of the total solids in the bioreactors during the successive cycles are shown in FIG. 4. The profiles of the TS were identical. Generally, the TS contents of the inoculum increased slightly from cycle to cycle likely due to the increase in the microbial biomass. The percentage of total solids reduction during the individual cycles was about 3±1.5.

Psychrophilic dry anaerobic digestion (PDAD) of cow feces with wheat straw bedding as ben demonstrated at 27% feed TS at OLR of 7.0 kg TCOD kg⁻¹ culture d⁻¹ g treatment cycle length (TCL) of 21 days. Cow feces plus wheat straw (27% TS at OLR 3.0 kg TCOD_{fed} kg⁻¹ culture d⁻¹ and TCL 21 days) yielded 182.9±16.9 _{N}L CH₄ kg⁻¹ VS fed and at OLR 6.0 kg TCOD kg⁻¹ culture d⁻¹ and TCL 21 days yielded 175±12 _{N}L CH₄ kg⁻¹ VS fed. VS-based substrate to inoculum ratio of 0.71 (equivalent to wet mass ratio of 0.37:1.0) is possible at OLR 6.0 kg TCOD m⁻³ d⁻¹. At OLR 3.0 kg TCOD_{fed} kg⁻¹ culture d⁻¹ PDAD of cow feces and wheat straw (TS 35%) yielded 188±17 _{N}L CH₄ kg⁻¹ VS fed during 21 days. Psychrophilic dry anaerobic digestion (PDAD) of cow feces as also been demonstrated herein at OLR of 8.0 kg TCOD kg⁻¹ culture d⁻¹ g and treatment cycle length (TCL) of 21 days with a specific methane yield of 140.7±11.1 _{N}L CH₄ kg⁻¹ VS fed.

The present disclosure will be more readily understood by referring to the following examples which are given to illustrate embodiments rather than to limit its scope.

### Example I

### Experimental Setup and Design

Six 40-L cylindrical barrels bioreactors were set-up and operated as pseudo sequential batch reactors (PSBR) at a hydraulic retention time (HRT) of 21 days in a temperature controlled room (20° C.). The laboratory scale sequencing batch reactors (SBR) used are as described in Canadian patent No. 2,138,091, the content of which is enclosed herewith by reference. The reactors were fitted with two gas lines; one for purging nitrogen gas immediately after feeding the substrate to maintain the anaerobic condition, and the second to release the biogas produced into the biogas volume measuring meter. The experimental design is given in Table 3.

TABLE 3
Experimental design

| | Retention time | | TS | Number of |
|---|---|---|---|---|
| Cycle | (days) | Substrate | (%) | replicates |
| 1 | 21 | CF + WS | 27 | 6 |
| | 42 | CF + WS | 27 | 6 |
| 2 | 21 | CF + WS | 27 | 6 |
| 3 to 12 | 21 | CF + WS | 27 | 3 |

Note:
CF = cow feces;
WS = wheat straw.

The purpose of the experiments was to assess CH₄ production from cow feces and wheat straw at feed total solids of 27% at psychrophilic conditions during a long-term study of repeated digestion cycles. A strategic objective was also to adapt the culture to ferment the substrate (cow feces and wheat straw) at psychrophilic conditions. The first cycle was extended to 42 days (2 HRT), while the cycles 2 to 12 were operated at 21 days (1 HRT). The mass of inoculum, feces, and/or straw fed to each bioreactor at the beginning of the successive cycles and the organic loading rate (OLR) are given in Table 4.

TABLE 4
Organic loading rate and total solids of the feed

| | | | | Solids | Substrate | | Organic loading | | | | |
|---|---|---|---|---|---|---|---|---|---|---|---|
| | | | | retention | to | | TCOD | VS | g TCOD | | |
| | Inoculum | Feces | Straw | time (SRT) | inoculum | Substrate | fed | fed | fed kg⁻¹_{inoc} (g | g VS | kg VS_{fed} |
| Cycle | (kg) | (kg) | (kg) | (days) | ratio (SIR) | TS (%) | (g) | (g) | TCOD kg⁻¹ inoc d⁻¹) | fed kg⁻¹_{inoc} | m⁻³ d⁻¹ |
| 1-3 | 6 | 0.865 | 0.20 | 139.3 | 0.18 | 27 | 347.0 | 267 | 57.8 (2.7) | 44.5 | 2.12 |
| 4 | 6 | 0.955 | 0.216 | 128.6 | 0.20 | 27 | 378.0 | 281 | 63.0 (3.0) | 49.5 | 2.36 |
| 5 | 6 | 0.794 | 0.206 | 147.0 | 0.16 | 27 | 378.0 | 323 | 63.0 (3.0) | 53.9 | 2.57 |
| 6 | 6 | 0.915 | 0.204 | 133.6 | 0.19 | 27 | 378.0 | 281 | 63.0 (3.0) | 46.8 | 2.23 |
| 7 | 6 | 0.815 | 0.204 | 144.6 | 0.17 | 27 | 378.0 | 281 | 63.0 (3.0) | 46.8 | 2.23 |
| 8 | 6 | 0.915 | 0.204 | 144.6 | 0.19 | 27 | 378.0 | 281 | 63.0 (3.0) | 46.8 | 2.23 |
| 9-11 | 6 | 0.884 | 0.20 | 137.2 | 0.18 | 27 | 378.0 | 269 | 63.0 (3.0) | 44.8 | 2.13 |
| 12 | 6 | 0.935 | 0.16 | 136.0 | 0.18 | 27 | 378.0 | 246 | 63.0 (3.0) | 41.0 | 1.95 |

Physico-chemical characteristics of the inoculum and substrates before feeding bioreactors were analyzed and are given in Table 5.

TABLE 5
Physicochemical characteristics of the inoculum, cow feces and mixed liquor at
the beginning of each digestion cycle

| | | | TCOD | TS | VS | Acetate | Propionate | Butyrate |
|---|---|---|---|---|---|---|---|---|
| Cycle | Substrate | pH | (g kg⁻¹) | (%) | (%) | (g kg⁻¹) | (g kg⁻¹) | (g kg⁻¹) |
| 1 | Inoculum | 7.42 ± 0.06 | — | 10.9 ± 0.9 | 9.2 ± 0.8 | 0.70 ± 0.49 | 0.21 ± 0.18 | 0.30 ± 0.06 |
| | Feces | 6.1 | 147.5 | 12.8 | 11.4 | 3.0 | 1.1 | 0.30 |
| | Mixed liquor | 7.22 ± 0.14 | — | 12.5 ± 0.49 | 10.9 ± 0.60 | 1.62 ± 0.90 | 0.48 ± 0.28 | 0.43 ± 0.25 |
| 2 | Inoculum | 7.3 ± 0.04 | — | 11.4 ± 0.18 | 9.6 ± 0.16 | 0.59 ± 0.44 | 0.22 ± 0.19 | 0.23 ± 0.05 |
| | Feces | 5.89 | 148.1 | 13.3 | 11.9 | 3.4 | 0.88 | 0.11 |
| | Mixed liquor | 7.0 ± 0.08 | — | 14.0 ± 0.2 | 12.2 ± 0.2 | 1.3 ± 0.00 | 0.38 ± 0.01 | 0.15 ± 0.01 |
| 3 | Inoculum | 7.3 ± 0.07 | — | 12.5 ± 0.1 | 11.0 ± 0.4 | 0.21 ± 0.01 | 0.02 ± 0.009 | 0.00 ± 0.00 |
| | Feces | 5.96 | 195.2 | 13.2 | 11.9 | 3.8 | 1.15 | 0.80 |
| | Mixed liquor | 7.1 | — | 15.4 ± 0.4 | 13.5 ± 0.4 | 1.9 ± 0.18 | 0.40 ± 0.06 | 0.11 ± 0.02 |
| 4 | Inoculum | 7.6 ± 0.02 | — | 13.3 ± 0.1 | 11.3 ± 0.1 | 0.25 ± 0.04 | 0.04 ± 0.06 | 0.2 ± 0.1 |
| | Feces | 6.64 | 173.5 | 13.2 | 11.8 | 3.9 | 1.0 | 0.65 |
| | Mixed liquor | 7.2 ± 0.1 | — | 15.4 ± 0.2 | 13.3 ± 0.2 | 1.5 ± 0.45 | 0.42 ± 0.16 | 0.23 ± 0.05 |
| 5 | Inoculum | 7.6 ± 0.02 | — | 13.3 ± 0.1 | 11.3 ± 0.1 | 0.25 ± 0.04 | 0.04 ± 0.06 | 0.2 ± 0.1 |
| | Feces | 6.5 | 183.2 | 12.8 | 11.3 | 3.4 | 1.5 | 0.24 |
| | Mixed liquor | 7.3 ± 0.04 | — | 15.9 ± 0.20 | 13.7 ± 0.2 | 1.10 ± 0.14 | 0.39 ± 0.15 | 0.27 ± 0.11 |
| 6 | Inoculum | 7.3 ± 0.1 | — | 15.4 ± 0.5 | 13.2 ± 0.5 | 0.15 ± 0.02 | 0.03 ± 0.00 | 0.02 ± 0.00 |
| | Feces | 6.5 | 183.2 | 12.8 | 11.3 | 3.4 | 1.5 | 0.24 |
| | Mixed liquor | 7.5 ± 0.4 | | 17.1 ± 0.3 | 14.9 ± 0.3 | 0.93 ± 0.04 | 0.21 ± 0.03 | 0.26 ± 0.02 |
| 7 | Inoculum | 7.3 ± 0.0 | | 15.3 ± 0.1 | 13.0 ± 0.3 | 0.26 ± 0.09 | 0.06 ± 0.02 | 0.08 ± 0.00 |
| | Feces | 7.0 | 184.2 | 12.5 | 11.2 | 1.5 | 0.46 | 0.26 |
| | Mixed liquor | 7.4 ± 0.3 | — | 17.0 ± 0.7 | 14.8 ± 0.6 | 0.76 ± 0.17 | 0.13 ± 0.03 | 0.07 ± 0.00 |
| 8 | Inoculum | 7.3 ± 0.0 | — | 15.8 ± 0.4 | 13.6 ± 0.4 | 0.22 ± 0.03 | 0.05 ± 0.02 | 0.22 ± 0.02 |
| | Feces | 7.0 | 184.2 | 12.5 | 11.2 | 1.5 | 0.46 | 0.26 |
| | Mixed liquor | 7.2 ± 0.1 | — | 17.8 ± 0.4 | 15.3 ± 0.3 | 1.12 ± 0.10 | 0.31 ± 0.03 | 0.41 ± 0.04 |
| 9 | Inoculum | 7.5 ± 0.0 | — | 16.1 ± 0.3 | 13.8 ± 0.3 | 0.25 ± 0.04 | 0.07 ± 0.03 | 0.18 ± 0.03 |
| | Feces | 7.0 | 184.2 | 12.5 | 11.2 | 1.5 | 0.46 | 0.26 |
| | Mixed liquor | 7.4 ± 0.1 | — | 17.9 ± 0.3 | 15.5 ± 0.6 | 0.86 ± 0.09 | 0.15 ± 0.03 | 0.52 ± 0.03 |
| 10 | Inoculum | 6.3 ± 1.6 | — | 16.6 ± 0.5 | 14.3 ± 0.5 | 0.23 ± 0.05 | 0.03 ± 0.0 | 0.14 ± 0.02 |
| | Feces | 6.8 | 220.5 | 16.2 | 14.3 | 2.4 | 1.3 | 0.7 |
| | Mixed liquor | 7.3 ± 0.1 | — | 18.8 ± 1.4 | 16.3 ± 1.1 | 0.96 ± 0.12 | 0.12 ± 0.05 | 0.35 ± 0.17 |
| 11 | Inoculum | 7.8 ± 0.1 | — | 16.9 ± 0.3 | 14.5 ± 0.2 | 0.13 ± 0.04 | 0.04 ± 0.03 | 0.03 ± 0.02 |
| | Feces | 6.8 | 220.5 | 16.2 | 14.3 | 2.4 | 1.3 | 0.7 |
| | Mixed liquor | 7.6 ± 0.1 | — | 18.5 ± 0.0 | 16.1 ± 0.9 | 0.76 ± 0.13 | 0.08 ± 0.04 | 0.11 ± 0.02 |
| 12 | Inoculum | 7.3 ± 0.1 | — | 16.7 ± 0.2 | 14.2 ± 0.2 | 0.12 ± 0.00 | 0.00 ± 0.00 | 0.02 ± 0.00 |
| | Feces | 6.8 | 220.5 | 16.2 | 14.3 | 2.4 | 1.3 | 0.7 |
| | Mixed liquor | 7.4 ± 0.1 | — | 18.9 ± 1.0 | 16.4 ± 1.0 | 1.0 ± 0.36 | 0.16 ± 0.09 | 0.19 ± 0.01 |

Note:
a- Wheat straw characteristics are: TS = 89%, VS = 85%, TCOD = 1097 g kg⁻¹.
b- TS of the substrate fed (cow feces and wheat straw) = 27%

The initial inoculum was obtained from a semi-industrial scale (11.4 m³; TS=9%) psychrophilic (20° C.) anaerobic reactor fed with fresh dairy manure (12% TS), and operated as a SBR. Fresh feces from dairy cows was collected at the experimental farm of the DSRDC. Feces were collected on wood boards, before getting in contact with urine and bedding, transferred into a plastic drum, stored at 4° C., before being fed to the reactors. Wheat straw was harvested at the DSRDC's experimental farm during fall 2011 and fall 2012 and chopped (3 mm) using a laboratory mill (Thomas Wiley Laboratory Mill Model 4, Arthur H. Thomas Company, Philadelphia, Pa.). Wheat straw and cow feces were mixed manually to obtain the desired substrate TS content (27%) while maintaining the design organic loading rate of 3.0 g TCOD kg⁻¹ culture day⁻¹.

Organic loading rate (OLR) has been calculated based on the masses of VS and TCOD of the substrate fed (Table 2). OLR was expressed in g of total VS fed per kg VS of inoculum, g of TCOD fed per kg of inoculum, and kg of total VS fed per m³ per day. The substrate to inoculum ratio (SIR; based on mass of VS) was 0.18±0.01 for all cycles.

Biogas volume produced was measured daily using calibrated wet tip gas meters while the biogas components (CH₄, H₂S, CO₂) were determined weekly using a Hach Carle 400 AGC gas chromatograph (GC) (Chandler Engineering, Houston, Tex.) at 85° C. with a helium gas flow rate of 30 mL The GC calibration was performed weekly with a standard gas (27.3% CO₂, 1.01% N₂, 71.16% CH₄, 0.53% H₂S). Methane production is reported in normalized liters (_{N}L CH₄). Total cumulative CH₄ yield was established at the end of each digestion cycle. Specific CH₄ yield in each cycle was calculated as the ratio of CH₄ produced over the mass of volatile solids (VS) fed to the reactor at the beginning of the cycle.

Samples were collected from each bioreactor and analyzed weekly for volatile fatty acids (VFAs), total solids (TS), volatile solids (VS), pH, and alkalinity. Total chemical oxygen demand (TCOD) was determined before and after each treatment cycle. TCOD, TS, VS, alkalinity and pH were determined using standard methods (APHA, 1992, “Standard methods for the examination of water and wastewater.”, 18 ed. American Public Health Association, Washington, D.C.). VFAs concentration was measured with a Perkin Elmer gas chromatograph model 8310 (Perkin Elmer, Waltham, Mass.), equipped with a DB-FFAP high resolution column.

The complex substrate (cow feces and wheat straw) were subjected to fiber analysis to determine their content of cellulose, hemicellulose, and lignin. Hemicellulose can be calculated as the difference between neutral detergent fiber (NDF) and acid detergent fiber (ADF), cellulose as the difference between acid detergent fiber and acid detergent lignin (ADL) (Bauer et al., 2009, Journal of Biotechnology, 142: 50-55).

The present psychrophilic dry anaerobic digestion (PDAD) described herein is the first process to digest solid materials that operates at psychrophilic temperature of about 20° C., between 10-25° C. (compared to know process that operate at approximately 32° C. to 55° C.). As seen in Table 2, the PDAD process described herein has the shortest treatment cycle length (21 days) and is applicable to different flow regime such as a batch process, plug flow process or even in a continuous/semi-continuous system.

The reactor/digester system used herein can be a plug flow type where the waste moves horizontally from one end to the other, the waste entering the digester which in turn, displaces digester volume, thereby causing an equal amount of material to exit from the digester.

The process described herein does not require mixing in the reactor to work and the reactor can be fed for example once a week or every two weeks. The process can further be used with existing manure handling equipment in order to minimise interference with farm operation. In addition, the PAD process described herein requires low energy input (no heating or mixing for example) and provides higher energy output than conventional anaerobic digestion processes.

### Example II

### Psychrophilic Dry Anaerobic Digestion of Cow Feces and Wheat Straw

A total of 25 bioreactors have been operated at various experimental conditions and a total of 103 treatment cycles (21 days each) have been completed during the period from 1 Apr. 2013 to 1 Mar. 2014 on cow feces and wheat straw (27 to 35% TS).

Multiple bioreactors have been setup and operated for each experimental design. The total number of bioreactors for all experimental conditions which have been examined during the reporting period (1 Apr. 2013 to 1 Mar. 2014) was 25 bioreactors. The total number of treatment cycles completed during the same reporting period was 103 (each treatment cycle is 21 days) for the various experimental conditions examined. Table 6 gives the details of all the experimental work which has been completed during the reporting period.

TABLE 6
Extensive outline of the experimental work for psychrophilic dry anaerobic
digestion.

| | | | | | Organic | |
|---|---|---|---|---|---|---|
| | | | | | loading | |
| | | | | | rate (kg | |
| Number of | | | | | TCOD m⁻³ | |
| treatment | Cow | Wheat | | | inoculum | Number of |
| Cycles | feces | straw | Silage | TS % | day⁻¹) | bioreactors |
| 2 | ✓ | ✓ | — | 27 | 3 | 6 |
| 12 | ✓ | ✓ | — | 27 | 3 | 3 |
| 7 | ✓ | ✓ | — | 27 | 4 | 3 |
| 7 | ✓ | ✓ | — | 27 | 5 | 4 |
| 7 | ✓ | ✓ | — | 27 | 6 | 2 |
| 11 | ✓ | ✓ | — | 35 | 3 | 3 |
| 10 | ✓ | ✓ | — | 35 | 4 | 2 |
| 10 | ✓ | ✓ | — | 35 | 5 | 2 |
| 4 | ✓ | — | — | 12-16 | 6 | 2 |
| 10 | ✓ | — | — | 12 | 7 | 2 |
| 7 | ✓ | — | — | 12 | 8 | 2 |
| 8 | | | Corn | 34 | 1-3 | 3 |
| 8 | | | Barley | 23 | 1-3 | 3 |
| 8 | | | Grass | 28 | 1-3 | 3 |
| 9 | ✓ | | Corn | 17 | 3 | 3 |
| 9 | ✓ | | Barley | 15 | 3 | 3 |
| 9 | ✓ | | Grass | 16 | 3 | 3 |

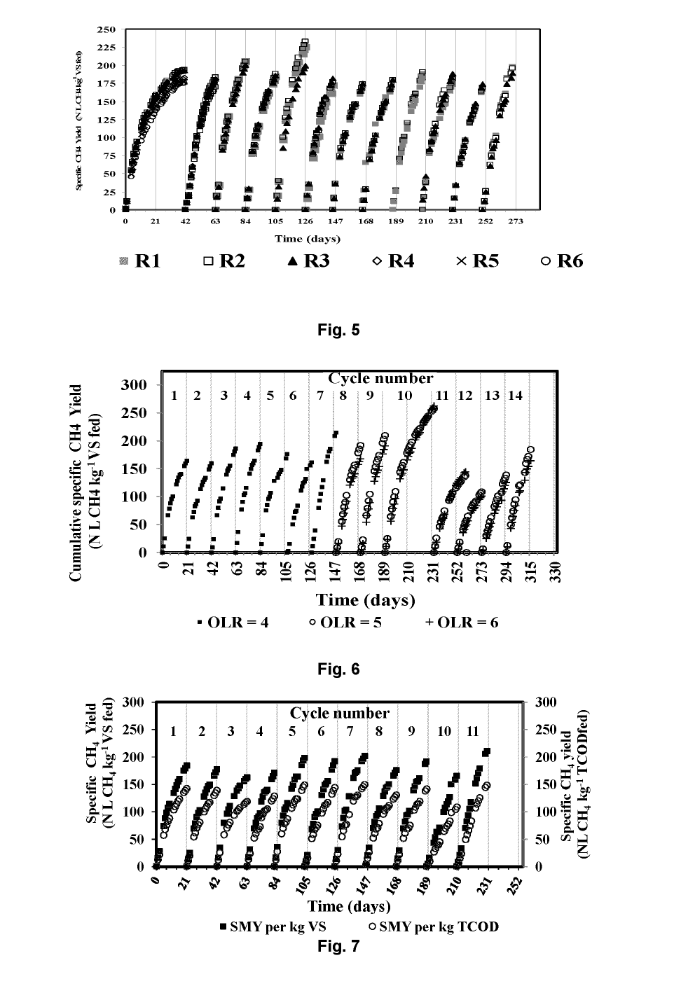

The results of long term (273 days comprising 12 successive cycles) of psychrophilic (20° C.) dry anaerobic digestion of cow feces and wheat straw in laboratory scale sequencing batch reactor inoculated with psychrophilic anaerobic mixed culture is shown in FIG. 5. An average specific methane yield (SMY) of 182.9±16.9 _{N}L CH₄ kg⁻¹ VS fed during the 12 successive cycles (273 days) has been achieved for feed total solids of 27% at organic loading rate 3.0 g TCOD kg⁻¹ culture day⁻¹ and hydraulic retention time of 21 days. A maximum SMY of 219.2±18 _{N}L CH₄ kg⁻¹ VS fed with a maximum CH₄ production rate of 10.2±0.8 _{N}L CH₄ kg⁻¹ VS day⁻¹ have been accomplished. The low levels of volatile fatty acids concentrations in the bioreactor indicated that hydrolysis was the reaction limiting step.

The results of long term (315 days experiment comprising 14 successive cycles) of psychrophilic (20° C.) dry anaerobic digestion (PDAD) of cow feces and wheat straw in laboratory scale sequence batch reactor operated at increasing organic loading rate is shown in FIG. 6. The PDAD process fed with a mixture of feces and straw (TS of 27%) over a treatment cycle length of 21 days at organic loading rate 4.0, 5.0 and 6.0 g TCOD kg⁻¹ inoculum d⁻¹ (3.9±0.1 and 4.4±0.1 kg VS kg⁻¹ inoculum d⁻¹) resulted in average specific methane yield (SMY) of 179.8±20.4, 163.6±39.5, 150.8±32.9 _{N}L CH₄ kg⁻¹ VS fed, respectively. PDAD of cow feces and wheat straw is possible with VS-based inoculum-to-substrate ratio of 1.4 at OLR of 6.0 g TCOD kg⁻¹ inoculum d⁻¹. Hydrolysis was the limiting step reaction.

The results of long term (231 days experiment comprising 11 successive cycles) of psychrophilic (20° C.) dry anaerobic digestion (PDAD) of cow feces and wheat straw at feed TS of 35% in laboratory scale sequence batch reactor operated at increasing organic loading rate is shown in FIG. 7. An average specific methane yield (SMY) of 188±17 _{N}L CH₄ kg⁻¹ VS fed during the 11 successive cycles (231 days) has been achieved with a maximum SMY of 214±14 _{N}L CH₄ kg⁻¹ VS fed (156±10 _{N}L CH₄ kg⁻¹ TCOD fed) and a maximum CH₄ production rate of 10.2±0.6 _{N}L CH₄ kg⁻¹ VS day⁻¹.

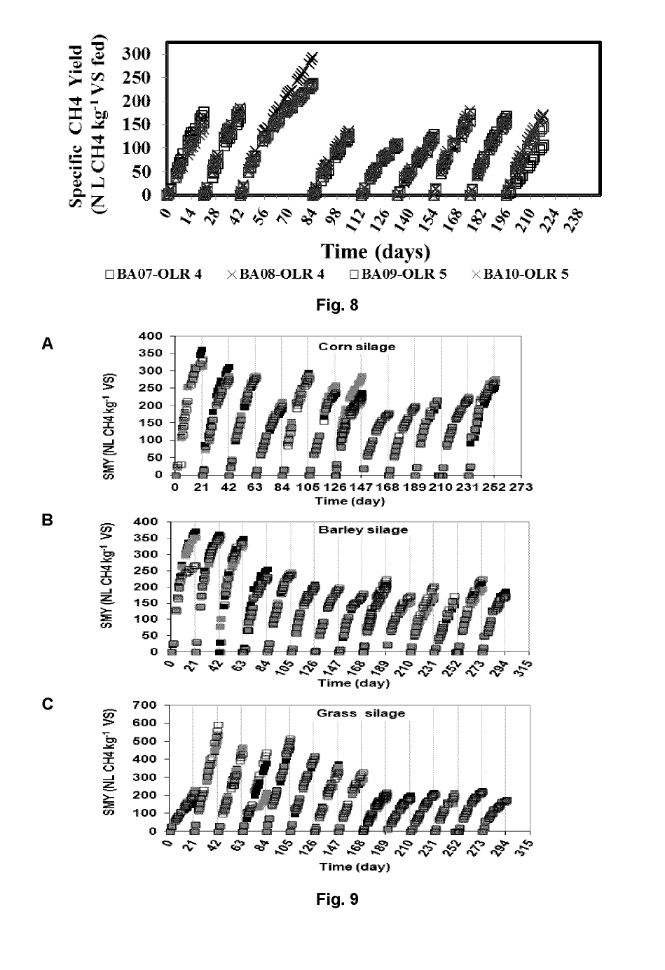

The results of long term (231 days experiment comprising 11 successive cycles) of psychrophilic (20° C.) dry anaerobic digestion (PDAD) of cow feces and wheat straw at feed TS of 35% in laboratory scale sequence batch reactor operated at increasing organic loading rate is shown in FIG. 8. An average specific methane yield (SMY) of 149.7±26.1 and 147.5±28.3 _{N}L CH₄ kg⁻¹ VS fed during the 11 successive cycles (231 days) has been achieved at OLR 4.0 and 5.0 g TCOD kg⁻¹ d⁻¹, respectively with a maximum SMY of 174.3±5 and 176.0±7.6 _{N}L CH₄ kg⁻¹ VS fed.

The results of long term (168 days experiment comprising 8 successive cycles) of psychrophilic (20° C.) dry anaerobic digestion (PDAD) of silages (corn, barley and grass) fed individually with cow feces at feed TS of 15 to 17% in laboratory scale sequence batch reactor operated at increasing organic loading rate is shown in FIG. 9. The ratio of silage to cow feces TCOD in feed has been kept at 1:4. An average specific methane yield (SMY) of 225.8±29.3, 186.7±18.0, 222.5±56.3 _{N}L CH₄ kg⁻¹ VS fed has been achieved with a maximum SMY of 252.9±27.3, 210.8±12.3, 229.3±9.2, _{N}L CH₄ kg⁻¹ VS fed for corn, barley, and crass silage with cow feces fed at OLR of 3.0 g TCOD kg⁻¹ inoculum d⁻¹, respectively.

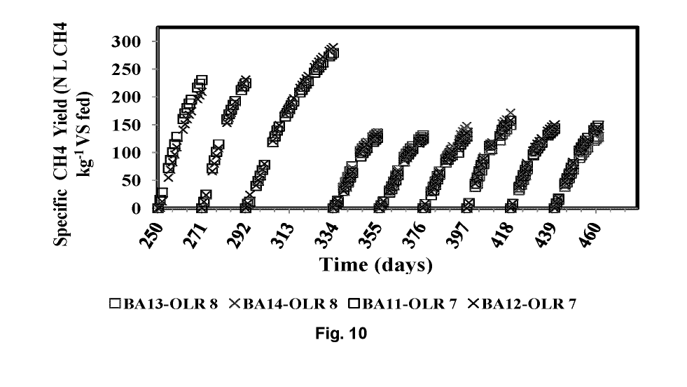

The results of long term (210 days experiment comprising 10 successive cycles) of psychrophilic (20° C.) dry anaerobic digestion (PDAD) of cow feces and wheat straw at feed TS of 27% in laboratory scale sequence batch reactor operated at increasing organic loading rate 7 and 8.0 g TCOD kg⁻¹ d⁻¹ is shown in FIG. 10. An average specific methane yield (SMY) of 162.5±38.6 and 140.7±11.1 _{N}L CH₄ kg⁻¹ VS fed have been achieved with a maximum SMY of 227.9±4.8 and 160.4±15.7 _{N}L CH₄ kg⁻¹ VS fed at OLR of 7 and 8.0 g TCOD kg⁻¹ d⁻¹, respectively.

While the invention has been described in connection with specific embodiments thereof, it will be understood that it is capable of further modifications and this application is intended to cover any variations, uses, or adaptations of the invention following, in general, and including such departures from the present disclosure as come within known or customary practice within the art and as may be applied to the essential features hereinbefore set forth, and as follows in the scope of the appended claims.

What is claimed is:

1. A process for the psychrophilic anaerobic digestion of high organic solids content waste comprising the steps of:
a) contacting the high organic solids content waste to an inoculum comprising psychrophilic anaerobic bacteria in a digester and
b) reacting the high organic solids content waste with the inoculum at a temperature below 25° C. to allow digestion of the high organic solids content waste.

2. The process of claim 1, wherein the high organic solids content waste is reacted with the inoculum at a temperature between 10 to 25° C.

3. The process of claim 1 or 2, wherein the high organic solids content waste is reacted with the inoculum at a temperature of 20° C.

4. The process of any one of claims 1-3, wherein the high organic solids content waste comprises between 12-45% of total solids content.

5. The process of any one of claims 1-4, wherein the high organic solids content waste is animal manure, energy crops, agri-food or municipal wastes.

6. The process of claim 5, wherein the animal manure is farm waste.

7. The process of claim 6, wherein the farm waste comprises a high fibrous content.

8. The process of claim 5 or 6, wherein the farm waste is dairy manure, beef manure, poultry manure, spoiled hay, silage or solid fraction of swine manure.

9. The process of claim 8, wherein the farm waste is cow manure.

10. The process of any one of claims 1-9, wherein the animal manure comprises cellulose, hemicellulose, lignin, fat and protein or a mixture thereof.

11. The process of any one of claims 1-10, comprising the further step of feeding the digester with an inoculum from the same digester of from a separate silo.

12. The process of claim 11, wherein the inoculum is feed continuously from the separate silo into the digester.

13. The process of any one of claims 1-12, wherein the inoculum is recuperated at the end of the digestion.

14. The process of any one of claims 1-13, wherein the digester is a batch reactor, a sequential batch reactor or a plug flow digester.

15. The process of any one of claims 1-14, wherein methane is recuperated during digestion of the high organic solids content waste.

16. The process of any one of claims 1-15, wherein a fertilizer is recuperated from the digester after digestion of the high organic solids content waste.

17. The process of any one of claims 1-16, wherein the high organic solids content waste is digested within a treatment cycle length of 21 days or less.
